(19) United States
(12) Patent Application Publication
(10) Pub. No.: US 2026/0259606 A1
(43) Pub. Date: Sep. 3, 2026

(54) Systems and Methods for Unsupervised Calibration of Brain-Computer Interfaces

(71) Applicant: The Board Of Trustees Of The Leland Stanford Junior University, Stanford, CA (US)

(72) Inventors: Francis R. Willett, Stanford, CA (US); Guy Wilson, Stanford, CA (US); Jaimie M. Henderson, Stanford, CA (US); Krishna Vaughn Shenoy, Stanford, CA (US); Shaul Druckmann, Stanford, CA (US)

(73) Assignee: The Board Of Trustees Of The Leland Stanford Junior University, Stanford, CA (US)

(21) Appl. No.: 18/995,763

(22) PCT Filed: Jul. 21, 2023
(86) PCT No.: PCT/US2023/070758

§ 371 Date: Jan. 16, 2025

Related U.S. Application Data
(60) Provisional application No. 63/369,060, filed on Jul. 21, 2022.

(51) Int. Cl. G06F 3/01; G06N 3/044; G06N 3/09

(52) U.S. Cl. CPC G06F 3/015; G06N 3/044; G06N 3/09

(57) ABSTRACT

Systems and methods for unsupervised calibration of brain-computer interfaces (BCIs) in accordance with embodiments of the invention are illustrated. One embodiment includes a closed-loop recalibrating (BCI) including a neural signal recorder configured to record brain activity, and a decoder, including a processor, and a memory, where the memory contains a neural decoder model, an inference model, and a decoder application that configures the processor to obtain a neural signal from the neural signal recorder, translate the neural signal into a command for an interface device communicatively coupled to the decoder, using the neural decoder model, infer an intended target of a user based on the command using the inference model, annotate the neural signal with the inferred intended target, and retrain the neural decoder model using the annotated neural signal as training data.

### CROSS-REFERENCE TO RELATED APPLICATIONS

The current application claims the benefit of and priority under 35 U.S.C. § 119(e) to U.S. Provisional Patent Application No. 63/369,060 entitled “Systems and Methods for Unsupervised Calibration of Brain-Computer Interfaces” filed Jul. 21, 2022. The disclosure of U.S. Provisional Patent Application No. 63/369,060 is hereby incorporated by reference in its entirety for all purposes.

### STATEMENT OF FEDERALLY SPONSORED RESEARCH

This invention was made with Government support under contract NSF GRFP DGE-1656518 awarded by the National Science Foundation. The Government has certain rights in the invention.

### FIELD OF THE INVENTION

The present invention generally relates to calibrating decoders for brain-computer interfaces, and enabling long-term unsupervised, closed-loop recalibration.

### BACKGROUND

Brain-computer interfaces (BCIs, also referred to as brain-machine interfaces, BMIs) are medical devices which record neural activity in a user's brain and translate said activity into machine instructions. A classic BCI use case is cursor control, where a user can control a virtual cursor on a monitor to control a computer.

BCIs can be built to record and utilize different types of neural activity. For example, electroencephalography (EEG) and electrocorticography (ECoG) signals can be used for minimally or less invasive recording. Some BCIs utilize and benefit from more spatially and/or temporally localized recording modalities, such as those produced by implantable intracortical microelectrode arrays. A commonly used microelectrode array is the Utah Array by Blackrock Neurotech of Salt Lake City, Utah. However, many intracortical microelectrode arrays have been developed and deployed for similar purposes.

### SUMMARY OF THE INVENTION

Systems and methods for unsupervised calibration of brain-computer interfaces (BCIs) in accordance with embodiments of the invention are illustrated. One embodiment includes a closed-loop recalibrating (BCI) including a neural signal recorder configured to record brain activity, and a decoder, including a processor, and a memory, where the memory contains a neural decoder model, an inference model, and a decoder application that configures the processor to obtain a neural signal from the neural signal recorder, translate the neural signal into a command for an interface device communicatively coupled to the decoder, using the neural decoder model, infer an intended target of a user based on the command using the inference model, annotate the neural signal with the inferred intended target, and retrain the neural decoder model using the annotated neural signal as training data.

In another embodiment, the decoder application further directs the processor to obtain an additional neural signal from the neural signal recorder, translate the additional neural signal into an additional command for the interface device using the retrained neural decoder model, and enact the additional command using the interface device.

In a further embodiment, the neural decoder model is a supervised machine learning model.

In still another embodiment, the neural signal recorder is an intracortical microelectrode array; an electrocorticography device; or an electroencephalography device.

In a still further embodiment, the inference model is a recurrent neural network.

In yet another embodiment, the decoder application further configures the processor to obtain a confidence value from the inference model indicating a predicted accuracy of the inferred intended target.

In a yet further embodiment, the closed-loop recalibrating BCI does not require manual recalibration when used at least every other day.

In another additional embodiment, the interface device is a computer providing a movable cursor in a 2-dimensional (2D) virtual environment.

In a further additional embodiment, the inference model is a hidden Markov model having: a description:

$${T}_{i , j} = P ( {H}_{t} = {h}_{i} \vert {H}_{t - 1} = {h}_{j} ) = \{ \begin{matrix}ϵ , & {h}_{i} = {h}_{j} \\ \frac{1 - ϵ}{N} , & else\end{matrix} ;$$

a posterior distribution:

$$P ( {O}_{t} = {v}_{t} , {p}_{t} \vert {H}_{t} = {h}_{j} ) = VonMises ( {θ}_{{v}_{t}} ; {κ}_{{d}_{{H}_{t}}} ) ;$$

and a priority probability:

$$( {c}_{t} \vert {H}_{t} ) = Bernoulli ( {c}_{t} ; f ( {‖ {p}_{t} - {H}_{t} ‖}_{2} ) .$$

In another embodiment again, the decoder application further directs the processor to obtain a plurality of neural signals from the neural signal recorder recorded during a predefined time window, translate each neural signal from the plurality of neural signals into a respective command for the interface device, using the neural decoder model, infer an intended target of a user based on each respective command, annotate each neural signal from the plurality of neural signals with the respective inferred intended target, and retrain the neural decoder model using the annotated plurality of neural signals.

In a further embodiment again, a closed-loop recalibration method for brain-computer interfaces (BCIs) includes obtaining a neural signal from a neural signal recorder, translating the neural signal into a command for an interface device communicatively coupled to the decoder, using a neural decoder model, inferring an intended target of a user based on the command using an inference model, annotating the neural signal with the inferred intended target, and retraining the neural decoder model using the annotated neural signal as training data.

In still yet another embodiment, obtaining an additional neural signal from the neural signal recorder, translating the additional neural signal into an additional command for the interface device using the retrained neural decoder model, and enacting the additional command using the interface device.

In a still yet further embodiment, the neural decoder model is a supervised machine learning model.

In still another additional embodiment, the neural signal recorder is an intracortical microelectrode array; an electrocorticography device; or an electroencephalography device.

In a still further additional embodiment, the inference model is a recurrent neural network.

In still another embodiment again, the method further includes obtaining a confidence value from the inference model indicating a predicted accuracy of the inferred intended target.

In a still further embodiment again, the neural signal decoder does not require manual recalibration when used at least every other day.

In yet another additional embodiment, the interface device is a computer providing a movable cursor in a 2-dimensional (2D) virtual environment.

In a yet further additional embodiment, the inference model is a hidden Markov model having: a description:

$${T}_{i , j} = P ( {H}_{t} = {h}_{i} \vert {H}_{t - 1} = {h}_{j} ) = \{ \begin{matrix}ϵ , & {h}_{i} = {h}_{j} \\ \frac{1 - ϵ}{N} , & else\end{matrix} ;$$

a posterior distribution:

$$P ( {O}_{t} = {v}_{t} , {p}_{t} \vert {H}_{t} = {h}_{j} ) = VonMises ( {θ}_{{v}_{t}} ; {κ}_{{d}_{{H}_{t}}} ) ;$$

and a priority probability:

$$( {c}_{t} \vert {H}_{t} ) = Bernoulli ( {c}_{t} ; f ( {‖ {p}_{t} - {H}_{t} ‖}_{2} ) .$$

In yet another embodiment again, the method further includes obtaining a plurality of neural signals from the neural signal recorder recorded during a predefined time window, translating each neural signal from the plurality of neural signals into a respective command for the interface device, using the neural decoder model, inferring an intended target of a user based on each respective command, annotating each neural signal from the plurality of neural signals with the respective inferred intended target, and retraining the neural decoder model using the annotated plurality of neural signals.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 is a PRI-T BCI system architecture diagram in accordance with an embodiment of the invention.

FIG. 2 is a block diagram for a PRI-T BCI decoder in accordance with an embodiment of the invention.

FIG. 3 is a flow chart of a PRI-T decoding process in accordance with an embodiment of the invention.

FIG. 4 is a graphical depiction of a PRI-T decoding process for cursor control in accordance with an embodiment of the invention.

### DETAILED DESCRIPTION

Brain-computer interfaces (BCIs) are critical devices for many who have lost motor functions. They have been successfully implanted in patients to restore speech, movement, and other natural freedoms. BCI systems typically include three main components: 1) a neural activity recording device such as (but not limited to) an electroencephalography (EEG) device, electrocorticography (ECoG) implant, or an implantable intracortical microelectrode array; 2) a decoder; and 3) the computer or machine to be interfaced with. A key problem for BCIs is that activity in the human brain changes over time, even for repeated tasks. This means that neural activity that should be translated to a respective control output by the decoder changes. Neural features exhibit drift over time, reflecting (among other causes) array movements, device degradation, physiological changes in single neurons, glial scarring, and the influence of varying behavior. This drift operates on multiple timescales. Single and multi-unit baseline firing rates can change even after a few minutes while the selectivity of their responses often vary on the order of days. As changes compound, decoders fit to a particular time period progressively worsen, resulting in a need for repeated recalibration.

Typically, a user on a daily basis must re-calibrate their BCI decoder by going through what can be a mentally rigorous, frustrating, and time-consuming process. Conventional recalibration requires a user to attempt to perform the same task over and over to reidentify the current neural activity associated with the task performance. The issue of recalibration is particularly pronounced for BCIs that use intracortical microelectrode arrays (iBCIs), as they are much more sensitive to small-scale changes in brain activity.

Recent efforts at building intrinsically robust decoders have shown promise counteracting these changes, but nevertheless only delay the time needed until recalibration is needed. Standard “manual” recalibration procedures require ground-truth target labels for supervised training; consequently, a BCI user has to carry out a predefined sequence of training examples and cannot engage in free use of their device during these times. Some BCIs have been proposed that use ECoG signals, which are measured using coarser electrode grids on the surface of the brain. Due to the larger electrode sizes compared to intracortical microelectrode arrays, there tends to be a robustness advantage over intracortical recordings. The averaging of many signals by the large electrode can mask the compounding variance, but the tradeoff in ECoG systems is less spatial resolution and low signal-to-noise ratio (SNR), which results in lower bandwidth BCI control.

In order to maintain the benefits of high-SNR intracortical recordings while mitigating signal instability, attempts have been made to utilize unsupervised recalibration methods for decoders. These are algorithms that recalibrate using the neural features only and do not require ground-truth knowledge of where the targets are located, which one was cued, or even the user's intentions. Recent work has focused on domain mapping strategies, where a function ƒ is sought such that the distribution of neural features from an unlabeled test set Pₜₑₛₜ matches those from a training period Pₜᵣₐᵢₙ when mapped through ƒ, i.e. ƒ(Pₜₑₛₜ)~Pₜᵣₐᵢₙ. In this case, the assumption is that with proper choice of ƒ, the subsequent mapping from features to targets is preserved. For noisy high-dimensional neural recordings, ƒ usually operates in a low-dimensional subspace that encodes a majority of task-related modulation.

In particular, factor analysis (FA) stabilization uses a FA model to identify task subspaces on two different days. A Procrustes realignment within these spaces is then used to realign new data so that old decoders, trained on the old subspace, can work. More recently, a method called ADAN (“adversarial domain adaptation network”) improved upon FA stabilization by leveraging deep learning for nonlinear alignment. A critical issue with these approaches is that using latent representations in neural data can cause a dimensionality bottleneck in decoder architectures that risks tossing out task-relevant information, and typically only update early components in a decoder (e.g. a single layer in FA stabilization, an autoencoder network with ADAN, and an alignment network in NoMAD).

Systems and methods described herein provide an alternative approach referred to as Probabilistic Retrospective Inference of Targets (PRI-T) to recalibration that is signal-agnostic, can arbitrarily retrain multi-layer decoder models, and outperforms existing recalibration models at long time scales. PRI-T decoders include an inference model in addition to a modified version of a conventional decoder model. As opposed to the prior approaches, instead of stabilizing the feature distribution of P(x) through a domain mapping strategy, P(y)—the prior knowledge of the task structure—is leveraged to infer user intentions during operation. Thus, PRI-T requires at least some understanding of the tasks that a user may wish to perform. For example, how a user may want to move a cursor in a 2d environment, how a user may want to move a robotic arm in a 3d environment, and/or any other task a user may wish to perform using their BCI. In numerous embodiments, multiple different decoders can be loaded onto a BCI platform that are built for specific tasks, which can then be swapped between by the user.

In numerous embodiments, PRI-T BCIs (i.e. BCIs that implement a PRI-T decoder) use a neural signal decoder similar to a standard BCI that decodes neural signals into computer commands. However, PRI-T BCIs also include an inference model that infers a goal the user intends to achieve based on the output of the neural signal decoder and the state of the environment. The inference model outputs the inferred goal along with a confidence metric reflecting the predicted accuracy of the inference model's inference. In many embodiments, the inferred goal and the confidence metric are used to annotate the input signal to the neural signal decoder that produced the output that the inference model operated upon for that given time step.

After a predefined period (e.g. every 30 seconds to 5 minutes), the annotated input signals are automatically used to recalibrate the neural signal decoder. In this way, the annotated input signals operate similar to a ground-truth training data set in a supervised machine learning environment, but do not necessarily reflect the ground-truth. As can be readily appreciated, the predefined training period can be varied based on the user, the task being performed, and any number of other variables as appropriate to the requirements of specific applications of embodiments of the invention. This constant, closed-loop recalibration results in a stable BCI decoder that can operate for months to years without significant degradation of performance. In many embodiments, PRI-T BCIs can maintain stable control over 30 days compared to a fixed decoder and compared to FA stabilization. PRI-T BCI architectures are discussed below, followed by a discussion of PRI-T decoding processes.

### PRI-T BCIs

PRI-T BCIs provide enhanced user experiences by removing the need for daily recalibration after a standard initial training period. In many embodiments, PRI-T BCIs can operate for months without the need for a calibration session that is visible to the user, especially when the PRI-T BCI is consistently used (e.g. on a daily or every other day basis). As distance between use periods increases over a day, there may be an increased chance of decoder performance degradation. PRI-T BCIs can be implemented using standard neural recording devices and programmable computing devices which implement decoders. While the below will discuss PRI-T BCIs predominantly with respect to intracortical microelectrode arrays as the neural signal recorder, as the inference model that is core to PRI-T functionality is agnostic to signal input, any number of different neural recording devices can be used such as, but not limited to, EEG and ECoG can be used as the neural signal recorder.

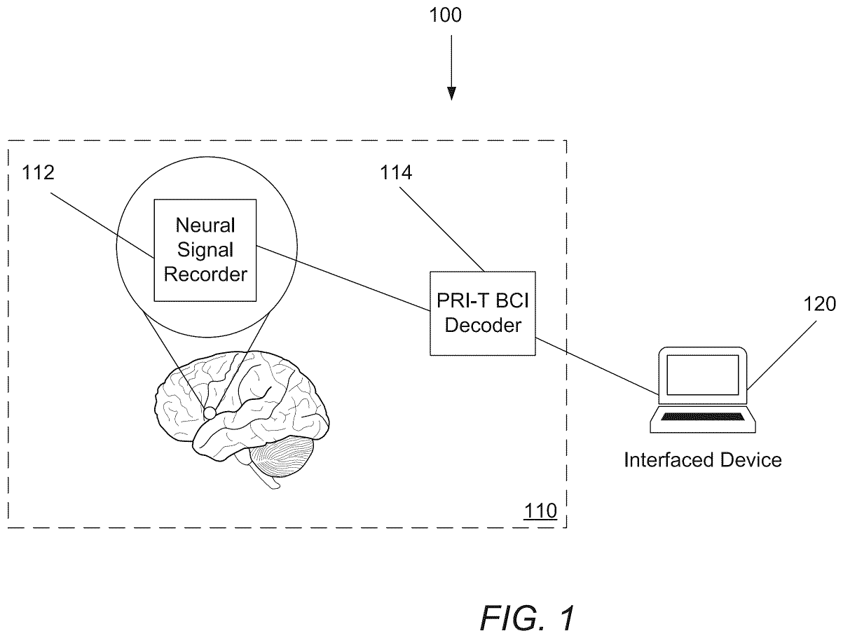

Turning now to FIG. 1, a PRI-T BCI system in accordance with an embodiment of the invention is illustrated. PRI-T BCI system 100 includes a PRI-T BCI 110 which is made up of a neural signal recorder 112 and a PRI-T BCI decoder 114. Illustrated neural signal recorder 112 is an intracortical microelectrode array implanted into a user's brain. However, as noted above, any number of different neural signal recording devices can be used as appropriate to the requirements of specific applications of embodiments of the invention. The PRI-T BCI decoder 114 is communicatively coupled to the neural signal decoder 114. In numerous embodiments, the PRI-T BCI decoder is a computing device capable of implementing the PRI-T decoding processes described herein. The PRI-T BCI decoder is communicatively coupled to an interfaced device 120. While a computer is depicted in FIG. 1 as the interfaced device, as can be readily appreciated, any number of different computing platforms or machines can be controlled using a BCI.

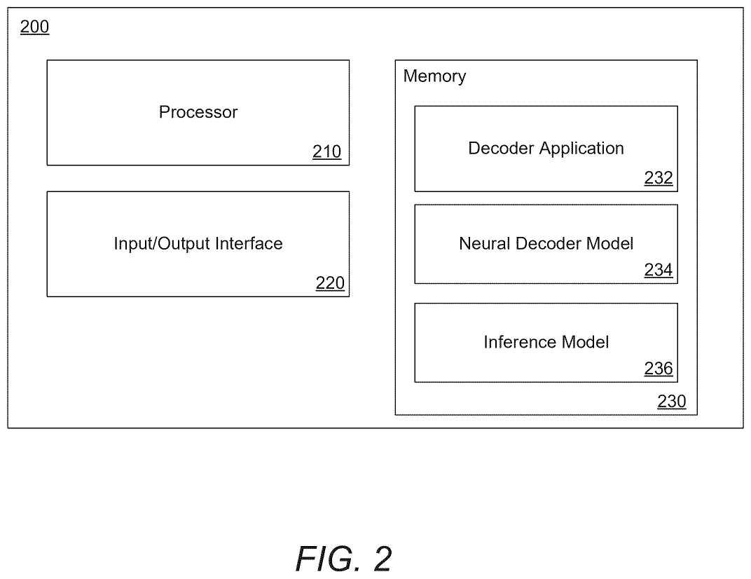

Turning now to FIG. 2, a PRI-T BCI decoder in accordance with an embodiment of the invention is illustrated. PRI-T BCI decoder 200 includes a processor 210. Processors can be any number of one or more types of logic processing circuits including (but not limited to) central processing units (CPUs), graphics processing units (GPUs), field-programmable gate arrays (FPGAs), application-specific integrated circuits (ASICs), and/or any other logic circuit capable of carrying out symbol decoding processes as appropriate to the requirements of specific applications of embodiments of the invention.

The PRI-T BCI decoder 200 further includes an input/output (I/O) interface 220. In numerous embodiments, I/O interfaces are capable of obtaining data from neural signal recorders and/or transmitting commands to interfaced devices. The PRI-T BCI decoder 200 further includes a memory 230. Memory can be volatile memory, nonvolatile memory, or any combination thereof. The memory 230 contains a decoder application 232. The decoder application is capable of directing at least the processor to decode neural activity into commands for interfaced devices using a neural decoder model 234. The decoder application is further capable of recalibrating the neural decoder model in a closed-loop, unsupervised manner using an inference model 236. In the illustrated embodiment, the neural decoder model and inference model are both stored in memory 230. However as can be readily appreciated, different components may be stored in different memory modules which are communicatively coupled to the processor as appropriate to the requirements of specific applications of embodiments of the invention.

While particular architectures are illustrated in FIGS. 1 and 2, one of ordinary skill would appreciate that any number of different computational architectures can be used without departing from the scope or spirit of the invention. For example, different neural recording modalities can be used, different interfaced devices can be used, different hardware platforms can be used to implement PRI-T BCI decoders, and/or any number of different architectural modifications can be made as appropriate to the requirements of specific applications of embodiments of the invention. PRI-T decoding processes are discussed in further detail below.

PRI-T Decoding Processes

PRI-T decoding processes are BCI decoding processes which utilize an inference model to continuously retrain the decoding model in order to provide a closed-loop, unsupervised recalibration process that does not impede the use of the BCI. As discussed above, the inference model is a model which is trained to predict targets or goals for given tasks given the output of the neural decoder model. Because many tasks tend to be identically repeatable over time, the inference model often only needs to be trained once for a given task. However, if the task space changes, the inference model itself may need to be retrained to the updated environment. By way of example, cursor control is a classic BCI task. Typically, cursors always behave in the same way; moving horizontally and/or vertically across a 2-dimensional plane (or additionally in depth within a 3-dimensional space).

Even for more complex tasks, they can often be broken down into more simple, repeatable sub-tasks. For example, picking up a block using a robotic arm could be broken down into moving the arm to the location of the block, grasping the block, and then moving the block. Additionally, in multi-step tasks such as constructing text strings or the aforementioned block example, state information can be used to predict future targets with relative certainty. Predictive text (often referred to as “autocomplete”) is a well known example of this.

Because different tasks can be very different, PRI-T decoding processes can include selection of the appropriate inference model, and optionally an appropriate neural decoder model appropriate for a given task that the user wishes to perform. The user can be provided a way to switch between different models, for example via a digital menu controllable by the PRI-T BCI, or via a physical input method if user motor ability allows.

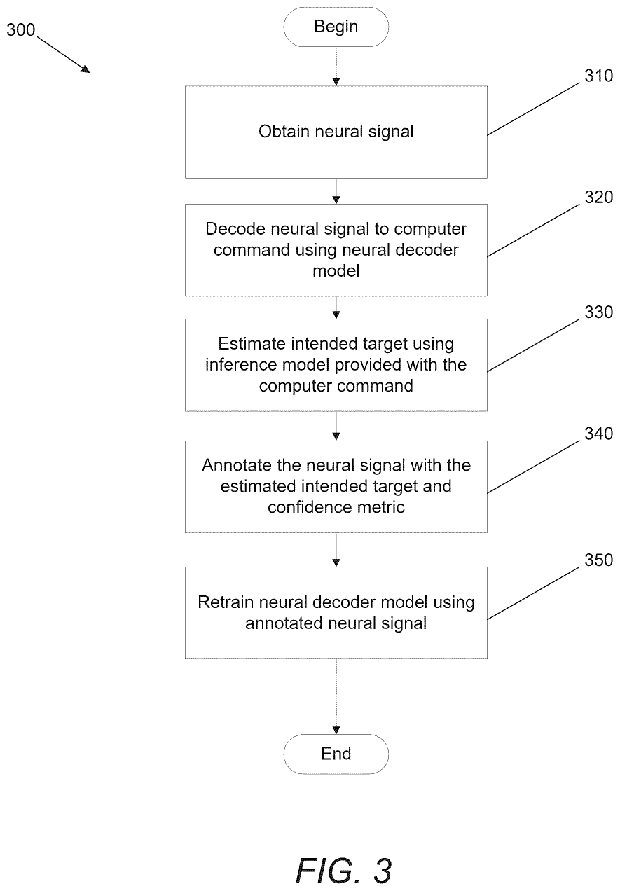

Turning now to FIG. 3, a PRI-T decoding process in accordance with an embodiment of the invention is illustrated. Process 300 includes obtaining (300) a neural signal from a neural signal recorder. The neural signals are decoded (320) to computer commands using a neural decoder model, similarly to a conventional BCI decoder. As can be readily appreciated, the computer command does not necessarily have to include all of the computational information needed to effect a change, but can instead be the information required by the interfaced device to implement the user's intention. By way of example, the computer command may be a command to move a virtual cursor to a given coordinate, just the coordinate, or a vector suggesting direction of movement for the virtual cursor. Indeed, the commands themselves can be anything required by the interfaced device to act in accordance with a user's direction.

An inference model is then used to estimate (330) an intended target of the user based on the output of the neural decoder model. In the above cursor example, this may be a given target cursor location. This is extendible to 3D coordinates for prosthetics or robot arms, as well as more complex tasks such as virtual avatar control, or any other arbitrary computer function. By way of further example, in a situation where the user is controlling a video game character, a whole host of abilities and actions may be available for the user to perform, which can be considered “targets”. Therefore, it is to be understood that inferred “targets” are not necessarily target coordinates, but can be any desired specific act in the context of the operating environment.

Inference models can be any number of different types of models that are able to relatively accurately model intention in a given environment. In many embodiments, inference models are generative models. For example, hidden Markov models (HMMs) are a useful model type for tasks which approximate a Markov process. In numerous embodiments, the inference model can be implemented using a recurrent neural network.

The neural signal which was provided as input to the neural decoder model is annotated (340) with the estimated intended target and a confidence metric which reflects a predicted likelihood that the estimated intended target is reflective of the actual intended target. The neural decoder model is then retrained (350) using the annotated neural signal as pseudo-ground truth training data. While, as mentioned above, the annotated neural signal is taking a similar role as actual ground truth data in a supervised machine learning system, this is considered to be an unsupervised training process due to the lack of actual ground-truth data.

In numerous embodiments, a batch of annotated neural signals are collected over a predefined time-window, and the model is trained on the batch instead of a single annotated neural signal. This process can occur over time windows as short as a few seconds, all the way up to a few minutes or an hour. While it is clear that the window could be extended even further, retraining on the order of minutes rather than hours or days can help account for small-scale changes in brain activity in intracortical systems. In numerous embodiments, the window can be extended when using ECoG or EEG-based neural signal recorders due to their higher robustness against small-scale change.

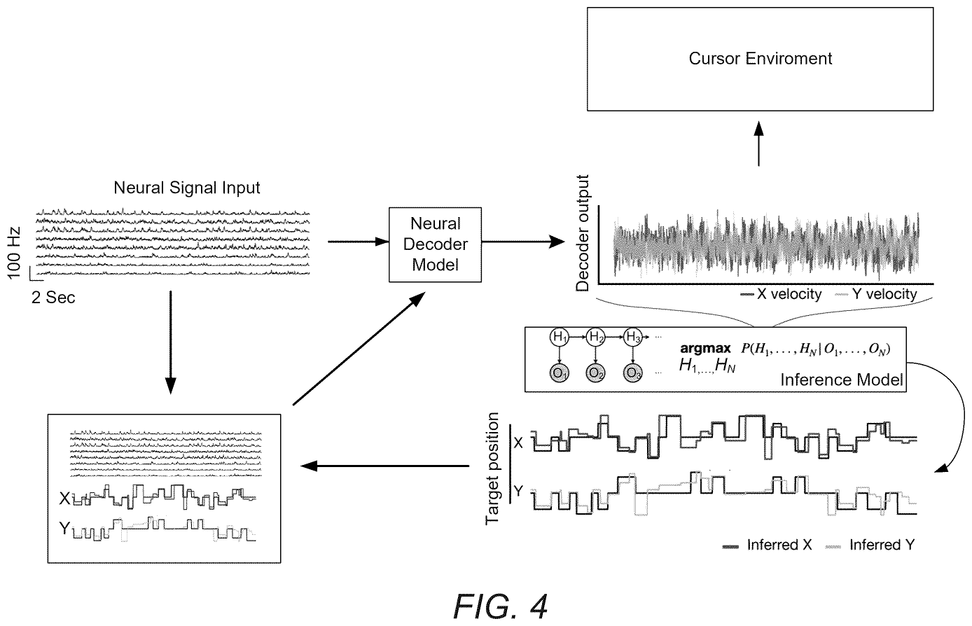

Turning now to FIG. 4, a specific implementation of a PRI-T decoding process for 2-dimensional cursor control using an HMM as the inference model in accordance with an embodiment of the invention is illustrated. As can be seen, the neural signal input is translated by the decoder into X and Y velocities and transmitted to a virtual keyboard where the cursor is moved in accordance with the velocities. At substantially the same time, the HMM infers a target X and Y coordinate based on the output of the decoder which is used to annotate the original neural signal input, which is subsequently used to retrain the decoder.

In the illustrated embodiment, the cursor position pₜ and velocity vₜ (collectively, observations Ot) at some timestep t is modeled as a reflection of the target position Hₜ using an HMM inference model. This has three components: 1) a description P(Hₜ|Hₜ₋₁) of how the target location evolves over time; 2) a posterior distribution P(Oₜ|Hₜ) over the observations with respect to a proposed target location; and 3) a prior probability P(Hₜ) of the target location.

When utilizing an HMM, it is useful if the underlying process approximates a Markov process. In the case of cursor control, it can be assumed that the target behaves according to discrete first-order Markov dynamics—the current (discrete) target location is simply a function of the previous state. This can be achieved by discretizing the screen containing the cursor into an N×N grid, and modeling the probability of moving from one grid position to another via a matrix:

$${T}_{i , j} = P ( {H}_{t} = {h}_{i} \vert {H}_{t - 1} = {h}_{j} ) = \{ \begin{matrix}ϵ , & {h}_{i} = {h}_{j} \\ \frac{1 - ϵ}{N} , & else\end{matrix}$$

This expression says that the target has some probability ϵ of remaining in the current location and a uniform probability of transitioning to any other location. The latter provides task generality as no knowledge is assumed of how the target location varies in any detail. For specific tasks such as keyboard typing, bigram probabilities can be leveraged here to improve within-task performance. In the example embodiment, ϵ=0.999 to reflect the timescale on which the HMM operates, and trials are on the order of a second whereas timesteps are approximately 20 ms. In many embodiments, these numbers can be varied depending on the user and the task at hand.

To model the observed cursor state as a function of the target location, a Von Mises distribution over cursor velocity angles is used. It is assumed that the cursor angle θᵥ_{ₜ} with respect to the current target should be concentrated around 0 degrees. This can provide an initial posterior of the form P(Oₜ|Hₜ)=VonMises(θᵥ_{ₜ}; κ) where κ controls the concentration of the distribution around its mean and reflects the noise in the decoder's angular outputs. Variability of cursor angle with respect to the target tends to increase as the cursor approaches the target. In many embodiments, κ can be parameterized as a function of the cursor-to-target distance in order to account for the variability:

$${d}_{{H}_{t}} = ‖ {p}_{t} - {H}_{t} ‖ :$$

$${κ}_{{d}_{{H}_{t}}} = {κ}_{0} * \frac{1}{1 + {e}^{- β ( {d}_{{H}_{t}} - {d}_{0} )}}$$

The initial kappa value κ₀ is weighted by a logistic function. At large distances, the effective κ is close to this value. At a smaller distance, κ is closer to 0. This causes a higher variance in the Von Mises distribution which means that noisier velocity angles are likely when near a target. The exponent and midpoint variables β and d₀ can be found via an exhaustive grid search resulting in a final posterior:

$$P ( {O}_{t} = {v}_{t} , {p}_{t} \vert {H}_{t} = {h}_{j} ) = VonMises ( {θ}_{{v}_{t}} ; {κ}_{{d}_{{H}_{t}}} )$$

Finally, a prior probability over the possible target states is obtained. This distribution P(Hₜ) reflects an a priori belief about the target location and can be used to encode task-specific regularities. For instance, in numerous embodiments, when optimizing for keyboard typing this could be the empirical distribution of starting letters across all words. To ensure generality, a uniform prior across all possible locations can be used instead. HMM hyperparameters can be selected using an automated tuning approach whereby hyperparameters which maximize the Viterbi probability are selected for use. In some embodiments, a more exhaustive optimization can be performed if desired.

In a variety of embodiments, click integration can be incorporated into the inference model for cursor control tasks. This can be achieved by integrating a click in the neural signal decoder's output through an indicator variable cₜ which indicates whether or nor a click occurred. To model this probabilistically, presume that clicks have a higher likelihood when the cursor is near the target:

$$( {c}_{t} \vert {H}_{t} ) = Bernoulli ( {c}_{t} ; f ( {‖ {p}_{t} - {H}_{t} ‖}_{2} )$$

where ƒ(⋅) is fit to the empirical click likelihood as a function of target distance from historical sessions. This probability is then multiplied with the Von Mises probability, yielding an overall posterior probability over the observations Oₜ={pₜ, vₜ, cₜ}.

Using the HMM structure, the inference model can then infer the most likely target sequence given the data P(H₁, . . . , Hₙ|O₁, . . . , Oₙ). Notably, this expression is an inversion of the posterior distribution above as it measures the likelihood of the target locations given the observed data. To do so, the exact inference can be performed for the most likely sequence of target locations using the Viterbi search algorithm. The Viterbi search algorithm's complexity is linear in sequence length, allowing for relatively fast computation. In numerous embodiments, the occupation probabilities (marginal probabilities of the target being in a given state at a given timestep given the observed data) during inference are obtained and used to weight the Viterbi labels. They can be obtained via the forward-backward algorithm. In some embodiments, the square of the maximal occupation probability at a given timestep are used as a weight, maxₕP(Hₜ=h|O₁, . . . , Oₙ), which may differ from the Viterbi sequence but can also improve performance.

As can be readily appreciated, while a specific implementation of an inference model is discussed above, any number of different inference model architectures can be used depending on the task to be performed. Further, different architectures can be used for the same task. By way of example, an RNN can be used instead of an HMM for a cursor control scenario. Indeed, any number of different inference models can be generated specific to a given task environment as appropriate to the requirements of specific applications of embodiments of the invention.

Although specific systems and methods for closed-loop, unsupervised recalibration of BCIs are discussed above, many different system architectures and methods can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A closed-loop recalibrating brain-computer interface (BCI), comprising:
a neural signal recorder configured to record brain activity; and
a decoder, comprising:
a processor; and
a memory, where the memory contains:
a neural decoder model;
an inference model; and
a decoder application that configures the processor to:
obtain a neural signal from the neural signal recorder;
translate the neural signal into a command for an interface device communicatively coupled to the decoder, using the neural decoder model;
infer an intended target of a user based on the command using the inference model;
annotate the neural signal with the inferred intended target; and
retrain the neural decoder model using the annotated neural signal as training data.

2. The closed-loop recalibrating BCI of claim 1, wherein the decoder application further directs the processor to:
obtain an additional neural signal from the neural signal recorder;
translate the additional neural signal into an additional command for the interface device using the retrained neural decoder model; and
enact the additional command using the interface device.

3. The closed-loop recalibrating BCI of claim 1, wherein the neural decoder model is a supervised machine learning model.

4. The closed-loop recalibrating BCI of claim 1, wherein the neural signal recorder is an intracortical microelectrode array; an electrocorticography device; or an electroencephalography device.

5. The closed-loop recalibrating BCI of claim 1, wherein the inference model is a recurrent neural network.

6. The closed-loop recalibrating BCI of claim 1, wherein the decoder application further configures the processor to obtain a confidence value from the inference model indicating a predicted accuracy of the inferred intended target.

7. The closed-loop recalibrating BCI of claim 1, wherein the closed-loop recalibrating BCI does not require manual recalibration when used at least every other day.

8. The closed-loop recalibrating BCI of claim 1, wherein the interface device is a computer providing a movable cursor in a 2-dimensional (2D) virtual environment.

9. The closed-loop recalibrating BCI of claim 8, wherein the inference model is a hidden Markov model having:
a description:
$${T}_{i , j} = P ( {H}_{t} = {h}_{i} \vert {H}_{t - 1} = {h}_{j} ) = \{ \begin{matrix}ϵ , & {h}_{i} = {h}_{j} \\ \frac{1 - ϵ}{N} , & else\end{matrix} ;$$
a posterior distribution:
$$P ( {O}_{t} = {v}_{t} , {p}_{t} \vert {H}_{t} = {h}_{j} ) = VonMises ( {θ}_{{v}_{t}} ; {κ}_{{d}_{{H}_{t}}} ) ;$$
and
a priority probability: (cₜ|Hₜ)=Bernoulli(cₜ; ƒ(∥pₜ−Hₜ∥₂).

10. The closed-loop recalibrating BCI of claim 1, wherein the decoder application further directs the processor to:
obtain a plurality of neural signals from the neural signal recorder recorded during a predefined time window;
translate each neural signal from the plurality of neural signals into a respective command for the interface device, using the neural decoder model;
infer an intended target of a user based on each respective command;
annotate each neural signal from the plurality of neural signals with the respective inferred intended target; and
retrain the neural decoder model using the annotated plurality of neural signals.

11. A closed-loop recalibration method for brain-computer interfaces (BCIs), comprising:
obtaining a neural signal from a neural signal recorder;
translating the neural signal into a command for an interface device communicatively coupled to the decoder, using a neural decoder model;
inferring an intended target of a user based on the command using an inference model;
annotating the neural signal with the inferred intended target; and
retraining the neural decoder model using the annotated neural signal as training data.

12. The closed-loop recalibration method of claim 11, further comprising:
obtaining an additional neural signal from the neural signal recorder;
translating the additional neural signal into an additional command for the interface device using the retrained neural decoder model; and
enacting the additional command using the interface device.

13. The closed-loop recalibration method of claim 11, wherein the neural decoder model is a supervised machine learning model.

14. The closed-loop recalibration method of claim 11, wherein the neural signal recorder is an intracortical microelectrode array; an electrocorticography device; or an electroencephalography device.

15. The closed-loop recalibration method of claim 11, wherein the inference model is a recurrent neural network.

16. The closed-loop recalibration method of claim 11, further comprising obtaining a confidence value from the inference model indicating a predicted accuracy of the inferred intended target.

17. The closed-loop recalibration method of claim 11, wherein the neural signal decoder does not require manual recalibration when used at least every other day.

18. The closed-loop recalibration method of claim 11, wherein the interface device is a computer providing a movable cursor in a 2-dimensional (2D) virtual environment.

19. The closed-loop recalibration method of claim 18, wherein the inference model is a hidden Markov model having:
a description:
$${T}_{i , j} = P ( {H}_{t} = {h}_{i} \vert {H}_{t - 1} = {h}_{j} ) = \{ \begin{matrix}ϵ , & {h}_{i} = {h}_{j} \\ \frac{1 - ϵ}{N} , & else\end{matrix} ;$$
a posterior distribution:
$$P ( {O}_{t} = {v}_{t} , {p}_{t} \vert {H}_{t} = {h}_{j} ) = VonMises ( {θ}_{{v}_{t}} ; {κ}_{{d}_{{H}_{t}}} ) ;$$
and
a priority probability: (cₜ|Hₜ)=Bernoulli(cₜ; ƒ(∥pₜ−Hₜ∥₂).

20. The closed-loop recalibration method of claim 11, further comprising:
obtaining a plurality of neural signals from the neural signal recorder recorded during a predefined time window;
translating each neural signal from the plurality of neural signals into a respective command for the interface device, using the neural decoder model;
inferring an intended target of a user based on each respective command;
annotating each neural signal from the plurality of neural signals with the respective inferred intended target; and
retraining the neural decoder model using the annotated plurality of neural signals.
